(19) United States
(12) Patent Application Publication
(10) Pub. No.: US 2026/0026835 A1
(43) Pub. Date: Jan. 29, 2026

(54) END EFFECTOR WITH SELECTIVELY REMOVED COATING FOR APPLYING ELECTRICAL ENERGY AND RELATED METHODS

(71) Applicant: Cilag GmbH International, Zug (CH)

(72) Inventors: Tyler N. Brehm, Arcanum, OH (US); Christopher M. Bull, Cincinnati, OH (US)

(21) Appl. No.: 18/784,271

(22) Filed: Jul. 25, 2024

(51) Int. Cl. A61B 17/32; A61B 17/00; A61B 18/00; A61B 18/14; C23C 16/02; C23C 16/22; C23C 16/455; C23C 16/50; C23C 16/56

(52) U.S. Cl. CPC A61B 17/320092; A61B 18/1445; C23C 16/02; C23C 16/22; C23C 16/45536; C23C 16/50; C23C 16/56; A61B 2017/00526; A61B 2017/00938; A61B 2017/320075; A61B 2017/320094; A61B 2018/00136; A61B 2018/00619; A61B 2018/0063; A61B 2018/1455

(57) ABSTRACT

A method of manufacturing a surgical instrument that includes a tissue contacting surface operable to apply ultrasonic energy or RF energy to tissue. The method includes applying a hydrophobic coating that includes silicone to a base surface of the tissue contacting surface to form an applied coating layer having a coating application thickness. The method also includes removing portions of the applied coating layer with a resiliently biased mechanical coating removal tool to form a plurality of removed portions of coating. A removal depth of each removed portion of the plurality of removed portions can each be from 50% to 100% of the coating application thickness such that from 10% to 50% of the applied coating layer is removed from the base surface.

### BACKGROUND

A variety of surgical instruments include a tissue cutting element and one or more elements that transmit radio frequency (RF) energy to tissue (e.g., to coagulate or seal the tissue). An example of such an electrosurgical instrument is the ENSEAL® Tissue Scaling Device by Ethicon Endo-Surgery, Inc., of Cincinnati, Ohio. Other examples of such devices and related concepts are disclosed in U.S. Pat. No. 7,354,440, entitled “Electrosurgical Instrument and Method of Use,” issued Apr. 8, 2008, the disclosure of which is incorporated by reference herein; U.S. Pat. No. 7,381,209, entitled “Electrosurgical Instrument,” issued Jun. 3, 2008, the disclosure of which is incorporated by reference herein.

Some instruments are capable of applying both ultrasonic energy and RF electrosurgical energy to tissue. Examples of such instruments are described in U.S. Pat. No. 9,949,785, entitled “Ultrasonic Surgical Instrument with Electrosurgical Feature,” issued Apr. 24, 2018, the disclosure of which is incorporated by reference herein; and U.S. Pat. No. 8,663,220, entitled “Ultrasonic Electrosurgical Instruments,” issued Mar. 4, 2014, the disclosure of which is incorporated by reference herein.

U.S. Pat. No. 9,272,095, entitled “Vessels, Contact Surfaces, and Coating and Inspection Apparatus and Methods,” issued on Mar. 1, 2016, relates to fabrication of coated contact surfaces of a medical device. U.S. Pat. No. 9,272,095 describes one utility for such a hydrophobic layer is to isolate a thermoplastic tube wall, made for example of polyethylene terephthalate (PET), from blood collected within the tube. A hydrophobic layer can be applied on top of a hydrophilic SiO₂ coating on the internal contact surface of the tube and the hydrophobic layer precursor can comprise hexamethyldisiloxane (HMDSO) or octamethylcyclotetrasiloxane (OMCTS). U.S. Pat. No. 9,272,095 does not appear to disclose hydrophobic coating being applied in addition to at least one of the microscopic surface pattern or the nanoscopic surface roughness.

U.S. Pub. No. 2014/0276407, entitled “Medical Devices Having Micropatterns,” published on Sep. 14, 2014, now abandoned, describes a plurality of nanostructures, a plurality of microstructures, and a plurality of hierarchical structures. A micropatterned polymer coating may be formed of any suitable material for a particular application, and may include one or more of a flexible polymer, a rigid polymer, a metal, an alloy, and any other material that may be suitable for a particular application. The micropatterned polymer coating could be applied by any of a wide variety of manufacturing techniques described herein including extrusion, compression dies, electro deposition, photoetching, or over molding configurations. U.S. Pub. No. 2014/0276407 does not appear to disclose a hydrophobic coating being applied in addition to at least one of the microscopic surface pattern or the nanoscopic surface roughness.

U.S. Pub. No. 2013/0138103 entitled “Electrosurgical Unit with Micro/nano Structure and the Manufacturing Method Thereof,” published on May 30, 2013, now abandoned, describes in FIG. 2 using the irradiation of the laser beam to directly construct a micro/nano structure on the surface of the blade while allowing the micro/nano structure to be composed of a hybrid of micro/nano elements. Referring to FIG. 3, the micro/nano structure 13 is formed directly on the blade 11. U.S. Pub. No. 2013/0138103 does not appear to disclose a hydrophobic coating in addition to the micro/nano structure.

U.S. Pat. No. 11,497,546 entitled “Area ratios of patterned coatings on RF electrodes to reduce sticking”, issued on Nov. 15, 2022, relates to an electrosurgical system includes an RF current generator, a handle body, and an end effector, wherein at least a portion of either a first or second energy delivery surface of the end effector, or both, may include one or more patterned coatings of an electrically non-conducting non-stick material. U.S. Pat. No. 11,497,546 teaches that the patterned coating may be formed from a coating of a material from which portions have been removed. U.S. Pat. No. 11,497,546 specifically teaches that an end mill is used to form elongated recessed features.

While a variety of surgical instruments have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary electrosurgical instrument;

FIG. 2 depicts a perspective view of an exemplary articulation assembly and end effector of the electrosurgical instrument of FIG. 1;

FIG. 3 depicts an exploded view of the articulation assembly and end effector of FIG. 2;

FIG. 4 depicts a perspective view of the end effector that of FIG. 2;

FIG. 5 depicts an exploded perspective view of the end effector of FIG. 2;

FIG. 6 depicts a top view of a jaw wherein a coating has been applied and subsequently selected portions of coating have been removed;

FIG. 7 depicts a perspective view of a drag bit utilized to remove selected portions of coating from the jaw of FIG. 6;

FIG. 8 depicts a cross-sectional view of a substrate with an applied coating with a drag bit being utilized to remove coating from substrate and an associated electrode pocket;

FIG. 9 depicts a cross-sectional view of a substrate with a coating having removed portions;

FIG. 10 depicts a cross-sectional view of a blaster blasting away removed portions from coating;

FIG. 11 depicts a cross-sectional view of a substrate with a coating having removed portions;

FIG. 12 depicts a cross-sectional view of a cryoblaster cryoblasting away removed portions from coating;

FIG. 13 depicts a cross-sectional view of a substrate with a coating having removed portions;

FIG. 14 depicts a cross-sectional view of a medium containing abrasive elements embedded on a surface prior to being clamped between two jaws to form removed portions in coating;

FIG. 15 depicts a cross-sectional view of a substrate with a coating having removed portions; and

FIG. 16 depicts a cross-sectional view of a medium containing abrasive elements embedded on a surface prior to be rolled across a substrate with a coating to form removed portions in coating.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

### I. Example of Electrosurgical Instrument

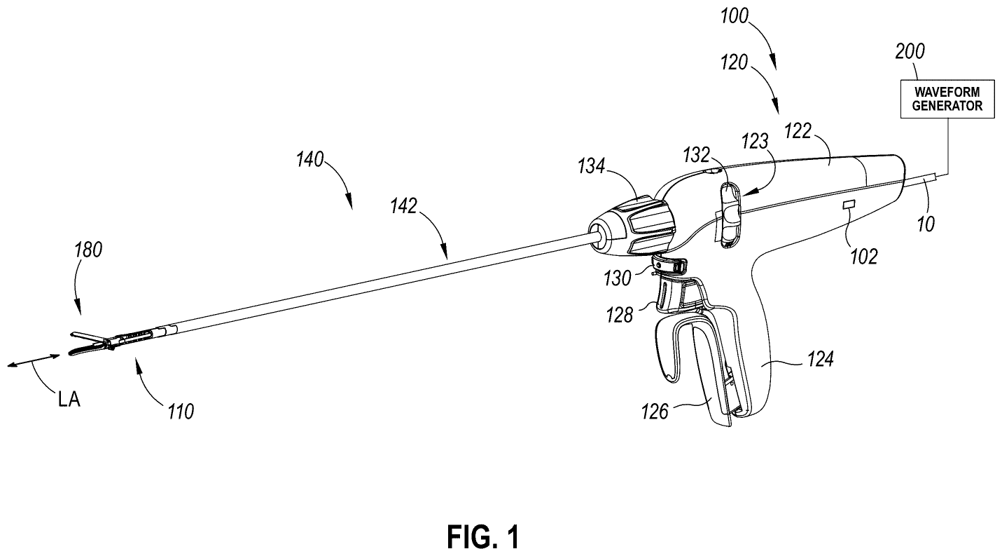

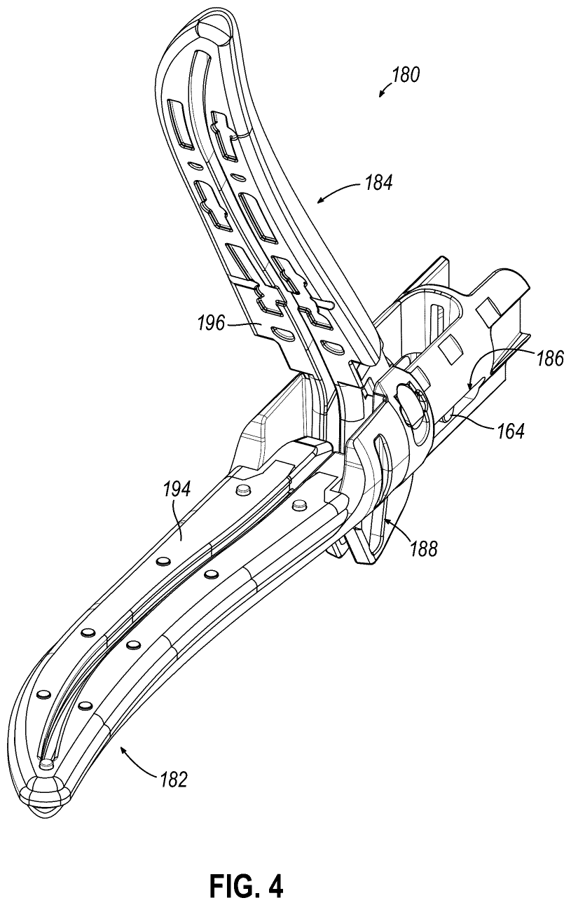

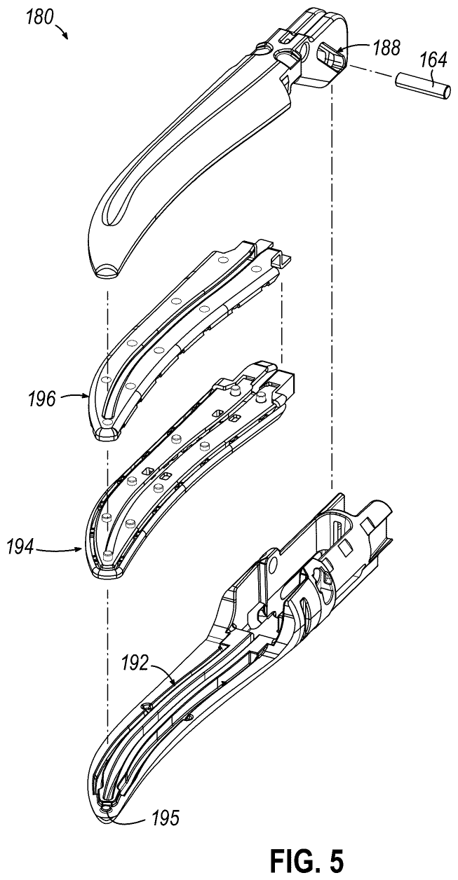

FIGS. 1-5 show a surgical system (98) including an exemplary electrosurgical instrument (100). As best seen in FIG. 1, electrosurgical instrument (100) includes a handle assembly (120), a shaft assembly (140), an articulation assembly (110), which may also be referred to as an articulation section (110), and an end effector (180). As will be described in greater detail below, end effector (180) of electrosurgical instrument (100) is operable to grasp, cut, and seal or weld tissue (e.g., a blood vessel, etc.). In this example, end effector (180) is configured to apply a non-therapeutic bipolar radio frequency (RF) energy in order to identify and/or verify that the correct tissue is present in the end effector such that a therapeutic RF energy can be applied to seal or weld tissue. However, it should be understood that electrosurgical instrument (100) may be configured to seal or weld tissue through any other suitable means that would be apparent to one skilled in the art in view of the teachings herein. For example, electrosurgical instrument (100) may be configured to seal or weld tissue via an ultrasonic blade, staples, etc. In the present example, electrosurgical instrument (100) is electrically coupled to a waveform generator (200) of surgical system (98), which is capable of delivering therapeutic and non-therapeutic energy, via power cable (10).

Waveform generator (200) may be configured to provide all or some of the electrical power requirements for use of electrosurgical instrument (100). Any suitable waveform generator (200) may be used as would be apparent to one skilled in the art in view of the teachings herein. By way of non-limiting example, the waveform generator (200) may be constructed in accordance with at least some of the teachings of U.S. Pat. No. 8,986,302, entitled “Surgical Generator for Ultrasonic and Electrosurgical Devices,” issued Mar. 24, 2015, the disclosure of which is incorporated by reference herein, in its entirety. While in the current example, electrosurgical instrument (100) is coupled to waveform generator (200) via power cable (10), electrosurgical instrument (100) may contain an internal power source or plurality of power sources, such as a battery and/or supercapacitors, to electrically power electrosurgical instrument (100). Of course, any suitable combination of power sources may be utilized to power electrosurgical instrument (100) as would be apparent to one skilled in the art in view of the teaching herein.

Handle assembly (120) is configured to be grasped by an operator with one hand, such that an operator may control and manipulate electrosurgical instrument (100) with a single hand. Although electrosurgical instrument (100) is primarily described herein as being used by a human user, it should be noted that alternative versions exist in which one or more robotic systems (e.g., a robotic arm) may be used to control and manipulate electrosurgical instrument (100). Shaft assembly (140) extends distally from handle assembly (120) and connects to articulation assembly (110). Articulation assembly (110) is also connected to a proximal end of end effector (180). As will be described in greater detail below, components of handle assembly (120) are configured to control end effector (180) such that an operator may grasp, cut, and seal or weld tissue. Articulation assembly (110) is configured to deflect end effector (180) from the longitudinal axis (LA) defined by shaft assembly (140).

Handle assembly (120) of the present example includes a control unit (102) housed within a body (122), a pistol grip (124), a jaw closure trigger (126), a knife trigger (128), an activation button (130), an articulation control (132), and a knob (134). As will be described in greater detail below, jaw closure trigger (126) may be pivoted toward and away from pistol grip (124) and/or body (122) to open and close jaws (182, 184) of end effector (180) to grasp tissue. Additionally, knife trigger (128) may be pivoted toward and away from pistol grip (124) and/or body (122) to actuate a knife member (176) within the confines of jaws (182, 184) to cut tissue captured between jaws (182, 184). Further, activation button (130) may be pressed to apply radio frequency (RF) energy to tissue via electrodes (194, 196) of jaws (182, 184), respectively. In some versions, electrodes (194, 196) of jaws (182, 184) are in a bifurcation configuration where electrodes (194, 196) move relative to a central axis and nearly equal and opposite to one another.

Body (122) of handle assembly (120) defines an opening (123) through which a portion of articulation control (132) protrudes. Articulation control (132) is rotatably disposed within body (122) such that an operator may rotate the portion of articulation control (132) protruding from opening (123) to rotate the portion of articulation control (132) located within body (122). Rotation of articulation control (132) relative to body (122) will bend articulation assembly (110) in order to drive deflection of end effector (180) from the longitudinal axis (LA) defined by shaft assembly (140). Articulation control (132) and articulation assembly (110) may include any suitable features to drive deflection of end effector (180) from the longitudinal axis (LA) defined by shaft assembly (140) as would be apparent to one skilled in the art in view of the teachings herein.

Knob (134) is rotatably disposed on the distal end of body (122) and is configured to rotate end effector (180), articulation assembly (110), and shaft assembly (140) about the longitudinal axis (LA) of shaft assembly (140) relative to handle assembly (120). While in the current example, end effector (180), articulation assembly (110), and shaft assembly (140) are rotated by knob (134), knob (134) may be configured to rotate end effector (180) and articulation assembly (110) relative to selected portions of shaft assembly (140). Knob (134) may include any suitable features to rotate end effector (180), articulation assembly (110), and shaft assembly (140) as would be apparent to one skilled in the art in view of the teachings herein.

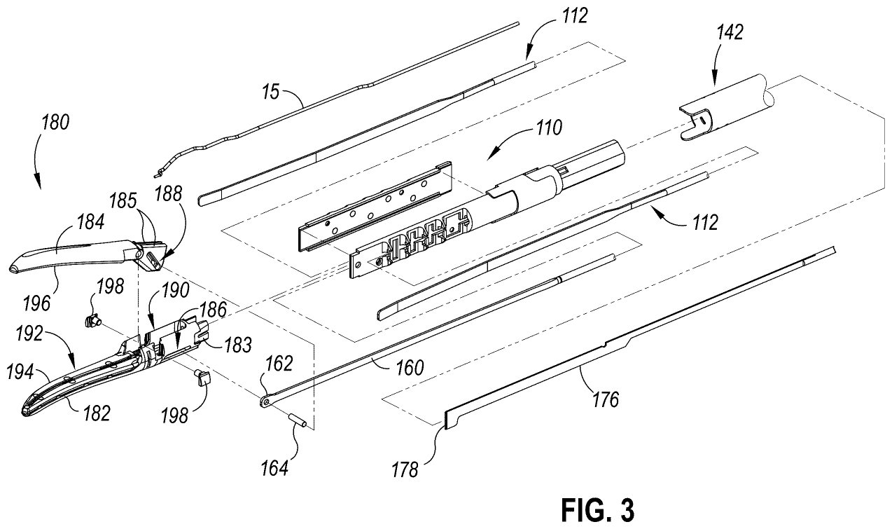

Shaft assembly (140) includes distal portion (142) extending distally from handle assembly (120) and a proximal portion housed within the confines of body (122) of handle assembly (120). Referring to FIG. 3, shaft assembly (140) houses a jaw closure connector (160) that couples jaw closure trigger (126) with end effector (180). Additionally, shaft assembly (140) houses a portion of knife member (176) extending between distal a distal cutting edge (178) of knife member (176) and knife trigger (128). Shaft assembly (140) also houses actuating members (112) that couple articulation assembly (110) with articulation control (132); as well as an electrical coupling (15) that operatively couples electrodes (194, 196) with activation button (130). As will be described in greater detail below, jaw closure connector (160) is configured to translate relative to shaft assembly (140) to open and close jaws (182, 184) of end effector (180); while knife member (176) is coupled to knife trigger (128) of handle assembly (120) to translate distal cutting edge (178) within the confines of end effector (180); and activation button (130) is configured to activate electrodes (194, 196).

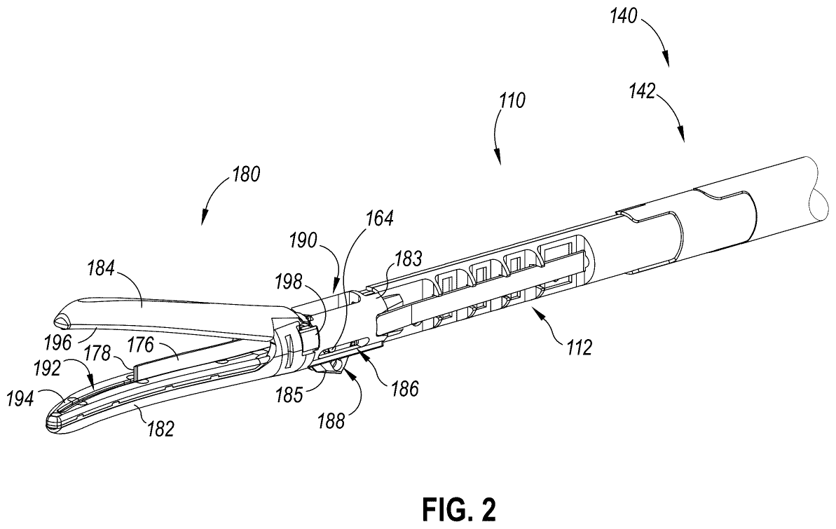

As best seen in FIGS. 2-5, end effector (180) includes lower jaw (182) pivotally coupled with upper jaw (184) via pivot couplings (198). Lower jaw (182) includes a proximal body (183) defining a slot (186), while upper jaw (184) includes proximal arms (185) defining a slot (188). Lower jaw (182) also defines a central channel (190) that is configured to receive proximal arms (185) of upper jaw (184), portions of knife member (176), jaw closure connector (160), and pin (164). Slots (186, 188) each slidably receive pin (164), which is attached to a distal coupling portion (162) of jaw closure connector (160). Additionally, lower jaw (182) includes a force sensor (195) located at a distal tip of lower jaw (182), though force sensor (195) may alternatively be positioned at any other suitable location. Force sensor (195) may be in communication with control unit (102). Force sensor (195) may be configured to measure the closure force generated by pivoting jaws (182, 184) into a closed configuration in accordance with the description herein. Additionally, force sensor (195) may communicate this data to control unit (102). Any suitable components may be used for force sensor (195) as would be apparent to one skilled in art in view of the teachings herein. For example, force sensor (195) may take the form of a strain gauge. In some variations, end effector (180) includes more than one force sensor.

While in the current example, a force sensor (195) is incorporated into electrosurgical instrument (100) and is in communication with control unit (102), any other suitable sensors or feedback mechanisms may be additionally or alternatively incorporated into electrosurgical instrument (100) while in communication with control unit (102) as would be apparent to one skilled in the art in view of the teachings herein. For instance, an articulation sensor or feedback mechanism may be incorporated into electrosurgical instrument (100), where the articulation sensor communicates signals to control unit (102) indicative of the degree end effector 180 is deflected from the longitudinal axis (LA) by articulation control (132) and articulation assembly (110).

As will be described in greater detail below, jaw closure connector (160) is operable to translate within central channel (190) of lower jaw (182). Translation of jaw closure connector (160) drives pin (164). As will also be described in greater detail below, with pin (164) being located within both slots (186, 188), and with slots (186, 188) being angled relative to each other, pin (164) cams against proximal arms (185) to pivot upper jaw (184) toward and away from lower jaw (182) about pivot couplings (198). Therefore, upper jaw (184) is configured to pivot toward and away from lower jaw (182) about pivot couplings (198) to grasp tissue.

The term “pivot” does not necessarily require rotation about a fixed axis and may include rotation about an axis that moves relative to end effector (180). Therefore, the axis at which upper jaw (184) pivots about lower jaw (182) may translate relative to both upper jaw (184) and lower jaw (182). Any suitable translation of the pivot axis may be used as would be apparent to one skilled in the art in view of the teachings herein.

Lower jaw (182) and upper jaw (184) also define a knife pathway (192). Knife pathway (192) is configured to slidably receive knife member (176), such that knife member (176) may be retracted, and advanced, to cut tissue captured between jaws (182, 184).

Lower jaw (182) and upper jaw (184) each comprise a respective electrodes (194, 196). The power source may provide RF energy to electrodes (194, 196) via electrical coupling (15) that extends through handle assembly (120), shaft assembly (140), articulation assembly (110), and electrically couples with one or both of electrodes (194, 196). Electrical coupling (15) may selectively activate electrodes (194, 196) in response to an operator pressing activation button (130). In some instances, control unit (102) may couple electrical coupling (15) with activation button (130), such that control unit (102) activates electrodes (194, 196) in response to operator pressing activation button (130). Control unit (102) may have any suitable components in order to perform suitable functions as would be apparent to one skilled in the art in view of the teachings herein. For instance, control unit (102) may have a processor, memory unit, suitable circuitry, etc. Examples of features and functionalities that may be incorporated into control unit (102) will be described in greater detail below.

As described above, jaw closure trigger (126) may be pivoted toward and away from pistol grip (124) and/or body (122) to open and close jaws (182, 184) of end effector (180) to grasp tissue. In particular, as will be described in greater detail below, pivoting jaw closure trigger (126) toward pistol grip (124) may proximally actuate jaw closure connector (160) and pin (164), which in turn cams against slots (188) of proximal arms (185) of upper jaw (184), thereby rotating upper jaw (184) about pivot couplings (198) toward lower jaw (182) such that jaws (182, 184) achieve a closed configuration.

In some versions, knife trigger (128) may be pivoted toward and away from body (122) and/or pistol grip (124) to actuate knife member (176) within knife pathway (192) of jaws (182, 184) to cut tissue captured between jaws (182, 184). In particular, handle assembly (120) further includes a knife coupling body that is slidably coupled along proximal portion of shaft assembly (140). Knife coupling body is coupled with knife member (176) such that translation of knife coupling body relative to proximal portion of shaft assembly (140) translates knife member (176) relative to shaft assembly (140).

In another version, knife coupling body may be coupled to a knife actuation assembly such that as knife trigger (128) pivots toward body (122) and/or pistol grip (124), knife actuation assembly drives knife coupling body distally, thereby driving knife member (176) distally within knife pathway (192). Because knife coupling body is coupled to knife member (176), knife member (176) translates distally within shaft assembly (140), articulation assembly (110), and within knife pathway (192) of end effector (180). Knife member (176) includes distal cutting edge (178) that is configured to sever tissue captured between jaws (182, 184). Therefore, pivoting knife trigger (128) causes knife member (176) to actuate within knife pathway (192) of end effector (180) to sever tissue captured between jaws (182, 184).

With distal cutting edge (178) of knife member (176) actuated to the advanced position, an operator may press activation button (130) to selectively activate electrodes (194, 196) of jaws (182, 184) to seal or weld severed tissue captured between jaws (182, 184). It should be understood that the operator may also press activation button (130) to selectively activate electrodes (194, 196) of jaws (182, 184) at any suitable time during exemplary use. Therefore, the operator may also press activation button (130) while knife member (176) is retracted. Next, the operator may release jaw closure trigger (126) such that jaws (182, 184) pivot into the opened configuration, releasing tissue.

### II. Coating Removal via Mechanical Resiliently Biased or Spring-Loaded Tool

### A. Overview

Instruments, such as instrument (100), may generate heat as end effectors, such as end effector (180), to seal and/or cut tissue. Tissue contacting surfaces of the instruments may tend to stick to the treated tissue. The tissue contacting surfaces are intended to include at least one of an ultrasonic blade, electrodes (194, 196), or another suitable tissue contacting surface. The tissue contacting surfaces include a base surface that is configured to contact the tissue. For example, base surfaces may include, for example, an outer surface of an ultrasonic blade or an electrode surface of electrodes (194, 196). Tissue sticking may cause reduced surgical efficiency.

The issue of tissue sticking is typically overcome by the addition of a coating layer to the tissue contacting surfaces of an instrument. However, the addition of a coating layer to tissue contacting surfaces may lead to tissue sealed and cut by the instrument to have low burst pressure. To improve the burst pressure while maintaining tissue anti-sticking performance, the present disclosure removes portions of the applied coating.

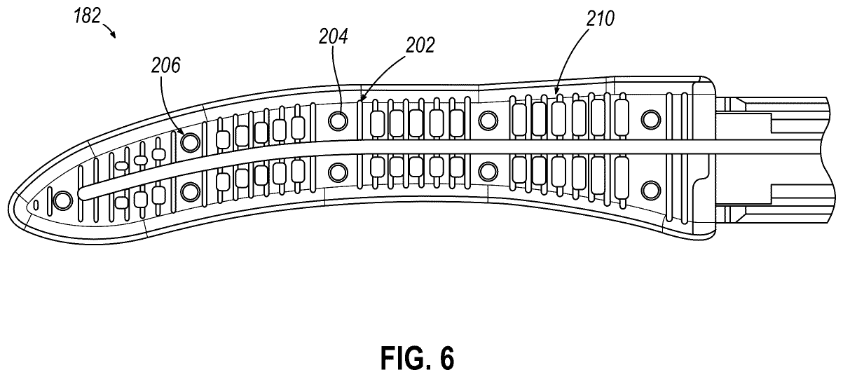

As will be described in greater detail below with reference to FIGS. 6-8, tissue contacting surfaces may include one or more removed portions (202) of any applied coating layer (206) to reduce sticking or otherwise promote tissue release while also improving the burst pressure of tissue samples prepared by tissue contacting surfaces. While removed portions (202) are described with reference to being applied to electrode surfaces of electrodes (194, 196), removed portions (202) can also be formed on an ultrasonic blade or another suitable surface that received a coating layer. As previously described, electrodes (194, 196) may be configured to cooperate to apply bipolar RF energy to tissue.

It is envisioned that removed portions (202) may be applied to select portions of the tissue contacting surfaces. Alternatively, removed portions (202) may be applied to the entire tissue contacting surface. In some versions, removed portions (202) may be applied to the entire outer surface of electrodes (194, 196) or the entire outer surface of an ultrasonic blade. In other versions, removed portions (202) may be applied to only select outer surfaces or to select portions of select outer surfaces of electrodes (194, 196) or select outer surfaces of an ultrasonic blade that experience sticking or high-pressure during tissue clamping. Removed portions (202) may be disposed on a metallic surface of the tissue contacting surface.

### B. Coating Methods

In one or more versions, a coating deposition method, such as, but not limited to, any form of chemical vapor deposition and/or any form of physical vapor deposition, such as, but not limited to, plasma vapor deposition (PVD), plasma enhanced chemical vapor deposition (PECVD), and/or atomic layer deposition (ALD). PECVD is a conformal coating process performed in a vacuum chamber which can apply a coating to any exposed areas of a substrate to the plasma/vapor during deposition. Plasma is used to activate precursor gases, allowing for lower deposition temperatures and higher deposition rates compared to conventional chemical vapor deposition techniques.

In one or more versions, PECVD may be utilized to apply a hydrophobic coating that includes silicone. In one or more versions, hydrophobic coatings that includes silicone can include polydimethylsiloxane-like (PDMS-like) coatings, polytetrafluoroethylene-like (PTFE-like) coatings, fluoropolymer coatings, silicone dip or spray coatings, and/or parylene coatings as coating layer (206). In one version, the coating is PDMS. PECVD shows very promising tissue anti-sticking results, however, the burst performance of thicker PDMS-like coatings (i.e., wherein the thickness is greater than 350 nm) at initial burst cycles can be low. In one or more versions, burst performance is determined by sealing and cutting tissue with instrument (100) discussed above, and then testing the performance of that seal versus varying amount of pressure. The greater burst performance equates to a better seal.

### C. Formation of Removed Portions

To address the low burst performance, portions of coating (206) applied during PECVD can be selectively removed as shown in FIG. 6. In one or more versions, the selective removal of portions of coating (206) can allow for the RF energy to pass through or be less impacted by coating (206) and as a result, the burst performance of a seal formed by instrument (100) containing coating (206) with removed portions (202) is improved as compared to the burst performance of a seal formed by instrument (100) containing coating (206) without removed portions (202). However, there is fine balance between improving the burst performance while maintaining the tissue anti-sticking performance of applied coating (206). FIG. 6 shows lower jaw (182) of exemplary electrosurgical instrument (100) wherein coating (206) has been applied and subsequently selected portions (202) of coating (206) have been removed. Although FIG. 6 shows lower jaw (182), upper jaw (184) can have coating (206) applied and subsequently have selected portions (202) of coating (206) removed. In the version of FIG. 6, removed portions (202) are removed in a pattern (210) running horizontally to the longitudinal axis of jaw (182), with each removed portion (202) having a diameter of 0.005 inches wherein each removed portion (202) is 0.020 inches apart from an adjacent removed portion (202).

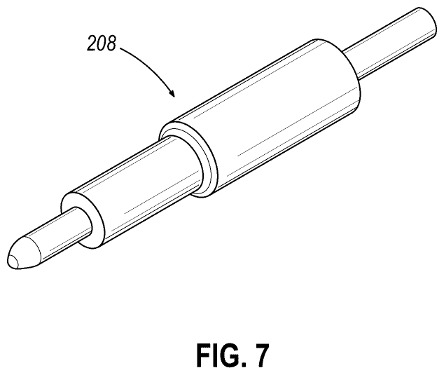

In one or more versions, portions of coating (206) applied during PECVD can be selectively removed utilizing a resiliently biased or spring-loaded mechanical coating removal tool, such as, but not limited to a drag bit (208), such as the one shown in FIG. 7. Drag bits, such as drag bit (208) are useful for fine line engraving, which allows for removed portions (202) to be created with more detail and precision, as opposed to using an end mill, which are used for drilling larger and less detailed holes. Drag bits, such as drag bit (208) can create patterns, such as coating removal pattern (210) on uneven surfaces by controlling the bias pressure of the tool. In one or more versions, controlled software designs using computer numerical control (CNC) in combination with drag bit (208) can create finely controlled and precise patterns, such as pattern (210) to match the curvature of the component from which coating (206) is being removed from.

In one or more versions, the process of selectively removing coating (206) takes place within a different vacuum chamber than the PECVD takes place. In one or more versions, PECVD can be applied in a batch process wherein multiple components are coated simultaneously and in one or more versions, the process of selectively removing coating (206) can occur in a batch process wherein multiple components have coating (206) removed simultaneously.

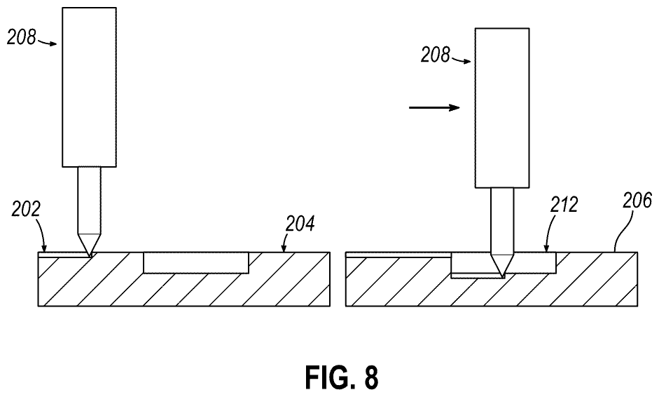

In one or more versions, the bias pressure acting on drag bit (208) can control the depth of the coating removal as well as providing the option to not remove coating from the electrode pockets. In yet other versions, the bias pressure acting on drag bit (208) will allow for drag bit (208) to travel into an electrode pocket, such as electrode pocket (212), remove coating (206), and then return to the surface on the other side of electrode pocket (212), such as shown in FIG. 8.

In one or more versions, drag bit (208) may have a diameter of ¼ of an inch, and in yet other versions, drag bit (208) may have a diameter of ½ of an inch. In one or more versions, drag bit (208) may have a length of between 2 inches and 5 inches, between 3 inches and 4 inches, or between 3.25 inches and 3.75 inches. In one or more versions, the length of drag bit (208) can have any value between any of the foregoing ranges. In one or more versions, drag bit (208) may include a 120-degree tip, and in yet other versions, drag bit (208) may include a 90-degree tip. In one or more versions, drag bit (208) may include a diamond tip.

### D. Characteristics of Removed Portions

By controlling the size and depth of each removed portion (202) relative to base substrate (204), removed portions (202) may decrease the amount of tissue sticking compared to base substrates (204) having generally smooth surfaces. For example, removed portions (202) may reduce tissue sticking compared to base substrates (204), which may reduce the number of protein bonding sites.

In one or more versions, each removed portion (202) has a removal depth of less than an entirety of a coating application thickness of applied coating (206) such that each removed portion (202) does not reach down to substrate (204) of lower jaw (182). In yet other versions, each removed portion (202) has a removal depth equal to the coating application thickness of applied coating (206), such that each removed portion (202) reaches down to substrate (204). In one or more versions, the removal depth of each removed portion (202) can be from about 50% to about 100% of the coating application thickness of coating (206). In yet other versions, the removal depth of each removed portion (202) may be from about 50% to about 90% of the coating application thickness of coating (206), from about 50% to about 80%, from about 50% to about 70%, and from about 50% to about 70%. In one or more versions, the removal depth of each removed portion (202) can have any value between any of the foregoing ranges.

In one or more versions, from about 10% to about 50% of applied coating (206) can be removed. In yet other versions, from about 10% to about 40% of applied coating (206) can be removed, from about 10% to about 30%, and from about 10% to about 20%. In one or more versions, the percentage of applied coating (206) removed can have any value between any of the foregoing ranges. In one or more versions, the percentage of applied coating (206) removed can be adjusted by increasing the number of removed portions (202), by increasing removal depth of each removed portion (202), by increasing the diameter of each removed portion (202), or combinations thereof.

Although removed portions (202) as shown in FIG. 6 have been removed in patterned formation (210), yet other versions removed portions (202) can be removed in an un-patterned formation. Although each removed portion (202) as shown in FIG. 6 is shown as a lined pattern (210), in yet other versions each removed portion (202) can take the form of other shapes such as, but not limited to circles, squares, rectangles, diamonds, or stars.

In one or more versions, coating layer (206) can have a coating application thickness (CT) of between about 200 nm and about 300 nm, between about 220 nm and about 280 nm, or between about 240 nm and about 260 nm. In one version, coating layer (206) may have a coating application thickness (CT) of greater than 300 nm. In one or more versions, coating layer (206) can have a coating application thickness between any of the foregoing ranges.

In one or more versions, the diameter of each removed portion (202) can be from about 0.001 inches to about 0.010 inches, from 0.002 inches to about 0.008 inches, or from about 0.004 inches to about 0.006 inches. In one or more versions, the diameter of each removed portion (202) can have any value between any of the foregoing ranges. In one version, the diameter of each removed portion (202) is about 0.005 inches.

In one or more versions, each removed portion (202) is from about 0.010 inches to about 0.030 inches apart from an adjacent removed portion (202), from about 0.012 inches to about 0.028 inches apart, from about 0.014 inches to about 0.026 inches apart, from about 0.016 inches to about 0.024 inches apart, or from 0.018 inches to about 0.022 inches apart. In one or more versions, the diameter of each removed portion (202) can have any value between any of the foregoing ranges. In one version, each removed portion (202) is about 0.020 inches apart from an adjacent removed portion (202).

### III. Coating Removal via Powder Blasting

### A. Overview

Instruments, such as instrument (100) may generate heat as end effectors, such as end effector (180), to seal and/or cut tissue. Tissue contacting surfaces of the instruments may tend to stick to the treated tissue. The tissue contacting surfaces are intended to include at least one of an ultrasonic blade, electrodes (194, 196), or another suitable tissue contacting surface. The tissue contacting surfaces include a base surface that is configured to contact the tissue. For example, base surfaces may include, for example, an outer surface of an ultrasonic blade or an electrode surface of electrodes (194, 196). Tissue sticking may cause reduced surgical efficiency.

The issue of tissue sticking is typically overcome by the addition of a coating layer to the tissue contacting surfaces of an instrument. However, the addition of a coating layer to tissue contacting surfaces may lead to tissue cut by the instrument to have low burst pressure. To improve the burst pressure while maintaining tissue anti-sticking performance, the present disclosure removes portions of the applied coating.

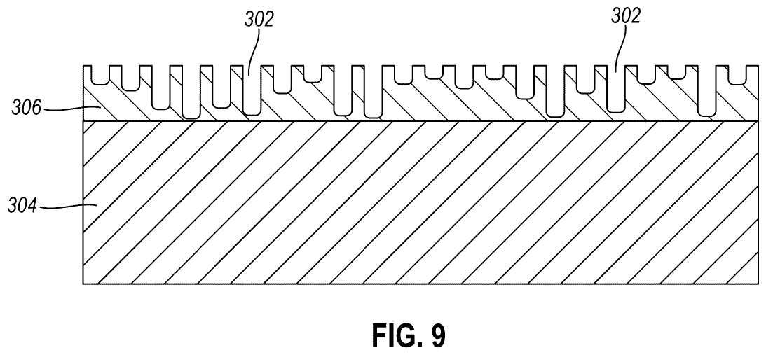

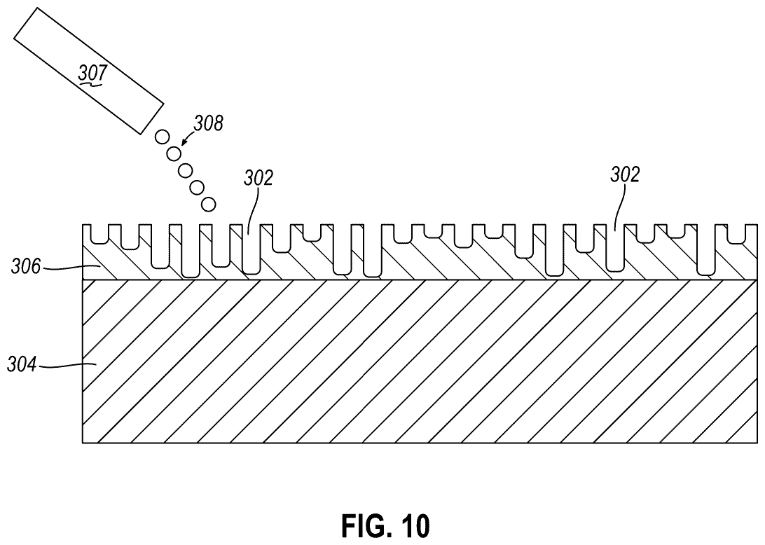

As will be described in greater detail below with reference to FIGS. 9 and 10, tissue contacting surfaces may include one or more removed portions (302) of any applied coating layer (306) to reduce sticking or otherwise promote tissue release while also improving the burst pressure of tissue samples prepared by tissue contacting surfaces. While removed portions (302) are described with reference to being applied to electrode surfaces of electrodes (194, 196), removed portions (302) can also be formed on an ultrasonic blade or another suitable surface that received a coating layer. As previously described, electrodes (194, 196) may be configured to cooperate to apply bipolar RF energy to tissue.

It is envisioned that removed portions (302) may be applied to select portions of the tissue contacting surfaces. Alternatively, removed portions (302) may be applied to the entire tissue contacting surface. In some versions, removed portions (302) may be applied to the entire outer surface of electrodes (194, 196) or the entire outer surface of an ultrasonic blade. In other versions, removed portions (302) may be applied to only select outer surfaces or to select portions of select outer surfaces of electrodes (194, 196) or select outer surfaces of an ultrasonic blade that experience sticking or high-pressure during tissue clamping. Removed portions (302) may be disposed on a metallic surface of the tissue contacting surface.

### B. Coating Methods

In one or more versions, a coating deposition method, such as, but not limited to, any form of chemical vapor deposition and/or any form of physical vapor deposition, such as, but not limited to, plasma vapor deposition (PVD), plasma enhanced chemical vapor deposition (PECVD), and/or atomic layer deposition (ALD). PECVD is a conformal coating process performed in a vacuum chamber which can apply a coating to any exposed areas of a substrate to the plasma/vapor during deposition. Plasma is used to activate precursor gases, allowing for lower deposition temperatures and higher deposition rates compared to conventional chemical vapor deposition techniques.

In one or more versions, PECVD may be utilized to apply a polymeric coating. In one or more versions, polymeric coatings can include polydimethylsiloxane-like (PDMS-like) coatings, polytetrafluoroethylene-like (PTFE-like) coatings, fluoropolymer coatings, silicone dip or spray coatings, and/or parylene coatings as coating layer (306). In one version, the coating is PDMS. PECVD shows very promising tissue anti-sticking results, however, the burst performance of thicker PDMS-like coatings (i.e., wherein the thickness is greater than 350 nm) at initial burst cycles can be low. In one or more versions, burst performance is determined by sealing and cutting tissue with instrument (100) discussed above, and then testing the performance of that seal versus varying amount of pressure. The greater burst performance equates to a better seal.

### C. Formation of Removed Portions

To address the low burst performance, portions of coating (306) applied during PECVD can be selectively removed as shown in FIG. 9. In one or more versions, the selective removal of portions of coating (306) can allow for the RF energy to pass through or be less impacted by coating (306) and as a result, the burst performance of a seal formed by instrument (100) containing coating (306) with removed portions (302) is improved as compared to the burst performance of a seal formed by instrument (100) containing coating (306) without removed portions (302). However, there is fine balance between improving the burst performance while maintaining the tissue anti-sticking performance of applied coating (306).

In one or more versions, portions of coating (306) applied during PECVD can be selectively removed utilizing blasting powders (308) being blasted from a blaster (307) into the coating (306), such as shown in FIG. 10. In one or more versions, the term blasting includes the acts of powder blasting, shot peening, or cold spraying of blasting powders (308) into coating (306) found on a substrate (304) to create removed portions (302). which coating (206) is being removed from.

In one or more versions, the process of selectively removing coating (306) takes place within a different vacuum chamber than the PECVD takes place. In one or more versions, PECVD can be applied in a batch process wherein multiple components are coated simultaneously and in one or more versions, the process of selectively removing coating (306) can occur in a batch process wherein multiple components have coating (306) removed simultaneously.

### D. Characteristics of Removed Portions

By controlling the size and depth of each removed portion (302) relative to base substrate (304), removed portions (302) may decrease the amount of tissue sticking compared to base substrates (304) having generally smooth surfaces. For example, removed portions (302) may reduce tissue sticking compared to base substrates (304), which may reduce the number of protein bonding sites.

In one or more versions, each removed portion (302) has a removal depth of less than an entirety of a coating application thickness of applied coating (306) such that each removed portion (302) does not reach down to substrate (304). In yet other versions, each removed portion (302) has a removal depth equal to the coating application thickness of applied coating (306), such that each removed portion (302) reaches down to substrate (304). In one or more versions, the removal depth of each removed portion (302) can be from about 50% to about 100% of the coating application thickness of coating (306). In yet other versions, the removal depth of each removed portion (302) may be from about 50% to about 90% of the coating application thickness of coating (306), from about 50% to about 80%, from about 50% to about 70%, and from about 50% to about 70%. In one or more versions, the removal depth of each removed portion (302) can have any value between any of the foregoing ranges.

In one or more versions, from about 10% to about 50% of applied coating (306) can be removed. In yet other versions, from about 10% to about 40% of applied coating (306) can be removed, from about 10% to about 30%, and from about 10% to about 20%. In one or more versions, the percentage of applied coating (306) removed can have any value between any of the foregoing ranges. In one or more versions, the percentage of applied coating (306) removed can be adjusted by increasing the number of removed portions (302), by increasing removal depth of each removed portion (302), by increasing the diameter of each removed portion (302), or combinations thereof.

In one or more versions, coating layer (306) can have a coating application thickness (CT) of between about 200 nm and about 300 nm, between about 220 nm and about 280 nm, or between about 240 nm and about 260 nm. In one version, coating layer (306) may have a coating application thickness (CT) of greater than 300 nm. In one or more versions, coating layer (306) can have a coating application thickness between any of the foregoing ranges.

In one or more versions, the diameter of each removed portion (302) can be from about 0.001 inches to about 0.010 inches, from 0.002 inches to about 0.008 inches, or from about 0.004 inches to about 0.006 inches. In one or more versions, the diameter of each removed portion (302) can have any value between any of the foregoing ranges. In one version, the diameter of each removed portion (302) is about 0.005 inches.

### E. Characteristics of Blasting Powder

In one or more versions, blasting powder (308) is selected from glass bead powder, ceramic bead powder, steel grit powder, silicon carbide powder, aluminum oxide powder, and crushed diamond powder.

In one or more versions, the blasting speed utilized to blast blasting powder (308) into coating layer (306) along with the powder size of blasting powder (308) will affect the removal depth of removed portions (302) along with the diameter of each removed portion (302). If the blasting speed is too slow, then blasting powder (308) may not penetrate coating layer (306), but if the blasting speed is too fast, then blasting powder (308) could damage substrate (304). If the powder size of blasting powder (308) is too small, the blasted coating layer (306) may be able to self-heal and close some or all of the removed portions (302), but if the powder size is too large, then too much of coating layer (306) could be removed.

### III. Coating Removal via Cryoblasting

### A. Overview

Instruments, such as instrument (100) may generate heat as end effectors, such as end effector (180), to seal and/or cut tissue. Tissue contacting surfaces of the instruments may tend to stick to the treated tissue. The tissue contacting surfaces are intended to include at least one of an ultrasonic blade, electrodes (194, 196), or another suitable tissue contacting surface. The tissue contacting surfaces include a base surface that is configured to contact the tissue. For example, base surfaces may include, for example, an outer surface of an ultrasonic blade or an electrode surface of electrodes (194, 196). Tissue sticking may cause reduced surgical efficiency.

The issue of tissue sticking is typically overcome by the addition of a coating layer to the tissue contacting surfaces of an instrument. However, the addition of a coating layer to a tissue contacting surface may lead to tissue cut by the instrument to have low burst pressure. To improve the burst pressure while maintaining tissue anti-sticking performance, the present disclosure removes portions of the applied coating.

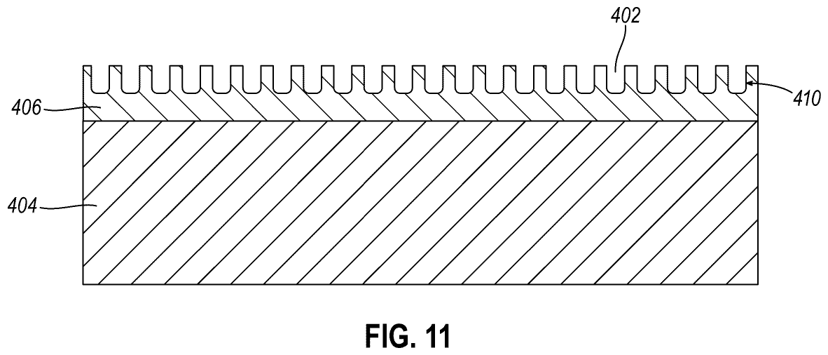

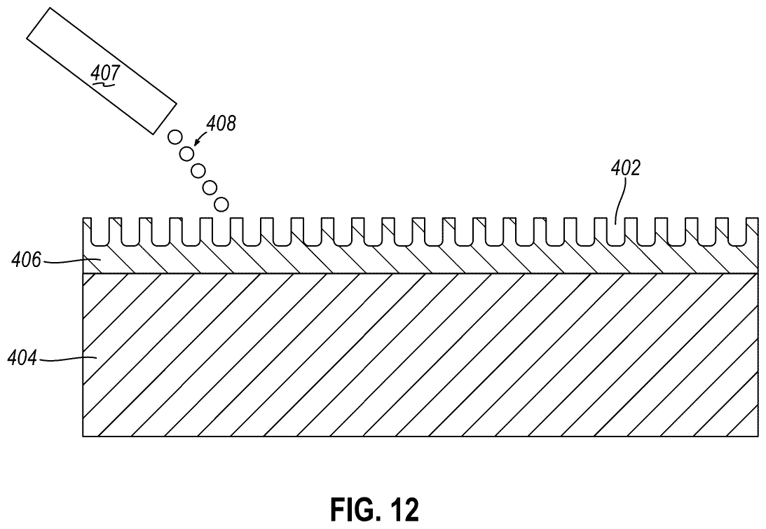

As will be described in greater detail below with reference to FIGS. 11 and 12, tissue contacting surfaces may include one or more removed portions (402) of any applied coating layer (406) to reduce sticking or otherwise promote tissue release while also improving the burst pressure of tissue samples prepared by tissue contacting surfaces. While removed portions (402) are described with reference to being applied to electrode surfaces of electrodes (194, 196), removed portions (402) can also be formed on an ultrasonic blade or another suitable surface that received a coating layer. As previously described, electrodes (194, 196) may be configured to cooperate to apply bipolar RF energy to tissue.

It is envisioned that removed portions (402) may be applied to select portions of the tissue contacting surfaces. Alternatively, removed portions (402) may be applied to the entire tissue contacting surface. In some versions, removed portions (402) may be applied to the entire outer surface of electrodes (194, 196) or the entire outer surface of an ultrasonic blade. In other versions, removed portions (402) may be applied to only select outer surfaces or to select portions of select outer surfaces of electrodes (194, 196) or select outer surfaces of an ultrasonic blade that experience sticking or high-pressure during tissue clamping. Removed portions (402) may be disposed on a metallic surface of the tissue contacting surface.

### B. Coating Methods

In one or more versions, a coating deposition method, such as, but not limited to, any form of chemical vapor deposition and/or any form of physical vapor deposition, such as, but not limited to, plasma vapor deposition (PVD), plasma enhanced chemical vapor deposition (PECVD), and/or atomic layer deposition (ALD). PECVD is a conformal coating process performed in a vacuum chamber which can apply a coating to any exposed areas of a substrate to the plasma/vapor during deposition. Plasma is used to activate precursor gases, allowing for lower deposition temperatures and higher deposition rates compared to conventional chemical vapor deposition techniques.

In one or more versions, PECVD may be utilized to apply a polymeric coating. In one or more versions, polymeric coatings can include polydimethylsiloxane-like (PDMS-like) coatings, polytetrafluoroethylene-like (PTFE-like) coatings, fluoropolymer coatings, silicone dip or spray coatings, and/or parylene coatings as coating layer (406). In one version, the coating is PDMS. PECVD shows very promising tissue anti-sticking results, however, the burst performance of thicker PDMS-like coatings (i.e., wherein the thickness is greater than 350 nm) at initial burst cycles can be low. In one or more versions, burst performance is determined by sealing and cutting tissue with instrument (100) discussed above, and then testing the performance of that seal versus varying amount of pressure. The greater burst performance equates to a better seal.

### C. Formation of Removed Portions

To address the low burst performance, portions of coating (406) applied during PECVD can be selectively removed as shown in FIG. 11. In one or more versions, the selective removal of portions of coating (406) can allow for the RF energy to pass through or be less impacted by coating (406) and as a result, the burst performance of a seal formed by instrument (100) containing coating (406) with removed portions (402) is improved as compared to the burst performance of a seal formed by instrument (100) containing coating (406) without removed portions (402). However, there is fine balance between improving the burst performance while maintaining the tissue anti-sticking performance of applied coating (406).

In one or more versions, portions of coating (406) applied during PECVD can be selectively removed utilizing a cryoblasting methodology, such as cryoblaster (407) to bombard coating layer (406) with carbon dioxide pellets (408) as shown in FIG. 12. In one or more version, carbon dioxide pellets (408) may be combined within other blasting media, such as, but not limited to glass bead powder, ceramic bead powder, steel grit powder, silicon carbide powder, aluminum oxide powder, and crushed diamond powder. In practice, carbon dioxide pellets (408) sublimate into a gas at the point of contact with coating layer (406) and due to this sublimation, no solid or liquid waste residue is experienced. In one or more versions, carbon dioxide pellets (408) are cryoblasted in a prescribed pattern to selectively form removed portions (402). In yet another embodiment, such as shown in FIGS. 11 and 12, carbon dioxide pellets (408) are cryoblasted in an unpatterned format to form removed portions 402. In one or more versions, areas of coating layer (406) intended to be preserved can be masked and protected from the cryoblasting. Another benefit of this method is that the masking should be able to last for a longer period than expected since there is no unwanted erosion of the mask or build-up of carbon dioxide pellets (408).

In one or more versions, the process of selectively removing coating (406) takes place within a different vacuum chamber than the PECVD takes place. In one or more versions, PECVD can be applied in a batch process wherein multiple components are coated simultaneously and in one or more versions, the process of selectively removing coating (406) can occur in a batch process wherein multiple components have coating (406) removed simultaneously.

### D. Characteristics of Removed Portions

By controlling the size and depth of each removed portion (402) relative to base substrate (404), removed portions (402) may decrease the amount of tissue sticking compared to base substrates (404) having generally smooth surfaces. For example, removed portions (402) may reduce tissue sticking compared to base substrates (404), which may reduce the number of protein bonding sites.

In one or more versions, each removed portion (402) has a removal depth of less than an entirety of a coating application thickness of applied coating (406) such that each removed portion (402) does not reach down to substrate (404). In yet other versions, each removed portion (402) has a removal depth equal to the coating application thickness of applied coating (406), such that each removed portion (402) reaches down to substrate (404). In one or more versions, the removal depth of each removed portion (402) can be from about 50% to about 100% of the coating application thickness of coating (406). In yet other versions, the removal depth of each removed portion (402) may be from about 50% to about 90% of the coating application thickness of coating (406), from about 50% to about 80%, from about 50% to about 70%, and from about 50% to about 70%. In one or more versions, the removal depth of each removed portion (402) can have any value between any of the foregoing ranges.

In one or more versions, from about 10% to about 50% of applied coating (406) can be removed. In yet other versions, from about 10% to about 40% of applied coating (406) can be removed, from about 10% to about 30%, and from about 10% to about 20%. In one or more versions, the percentage of applied coating (406) removed can have any value between any of the foregoing ranges. In one or more versions, the percentage of applied coating (406) removed can be adjusted by increasing the number of removed portions (402), by increasing removal depth of each removed portion (402), by increasing the diameter of each removed portion (402), or combinations thereof.

Although removed portions (402) as shown in FIG. 11 have been removed in patterned formation (410), yet other versions removed portions (402) can be removed in an un-patterned formation. Although each removed portion (402) as shown in FIG. 11 is shown as a circular dot pattern (410), in yet other versions each removed portion (402) can take the form of other shapes such as, but not limited to circles, squares, rectangles, diamonds, or stars.

In one or more versions, coating layer (406) can have a coating application thickness (CT) of between about 200 nm and about 300 nm, between about 220 nm and about 280 nm, or between about 240 nm and about 260 nm. In one version, coating layer (406) may have a coating application thickness (CT) of greater than 300 nm. In one or more versions, coating layer (406) can have a coating application thickness between any of the foregoing ranges.

In one or more versions, the diameter of each removed portion (402) can be from about 0.001 inches to about 0.010 inches, from 0.002 inches to about 0.008 inches, or from about 0.004 inches to about 0.006 inches. In one or more versions, the diameter of each removed portion (402) can have any value between any of the foregoing ranges. In one version, the diameter of each removed portion (402) is about 0.005 inches.

### E. Characteristics of Carbon Dioxide Pellets

In one or more versions, the blasting speed utilized to blast carbon dioxide pellets (408) into coating layer (406) along with the powder size of carbon dioxide pellets (408) will affect the removal depth of removed portions (402) along with the diameter of each removed portion (402). If the blasting speed is too slow, then carbon dioxide pellets (408) may not penetrate coating layer (406), but if the blasting speed is too fast, then carbon dioxide pellets (408) could damage substrate (404). If the powder size of carbon dioxide pellets (408) is too small, the blasted coating layer (406) may be able to self-heal and close some or all of the removed portions (402), but if the powder size is too large, then too much of coating layer (406) could be removed.

### IV. Coating Removal via Mechanically Applied Abrasion

### A. Overview

Instruments, such as instrument (100) may generate heat as end effectors, such as end effector (180), to seal and/or cut tissue. Tissue contacting surfaces of the instruments may tend to stick to the treated tissue. The tissue contacting surfaces are intended to include at least one of an ultrasonic blade, electrodes (194, 196), or another suitable tissue contacting surface. The tissue contacting surfaces include a base surface that is configured to contact the tissue. For example, base surfaces may include, for example, an outer surface of an ultrasonic blade or an electrode surface of electrodes (194, 196). Tissue sticking may cause reduced surgical efficiency.

The issue of tissue sticking is typically overcome by the addition of a coating layer to the tissue contacting surfaces of an instrument. However, the addition of a coating layer to tissue contacting surfaces may lead to tissue cut by the instrument to have low burst pressure. To improve the burst pressure while maintaining tissue anti-sticking performance, the present disclosure removes portions of the applied coating.

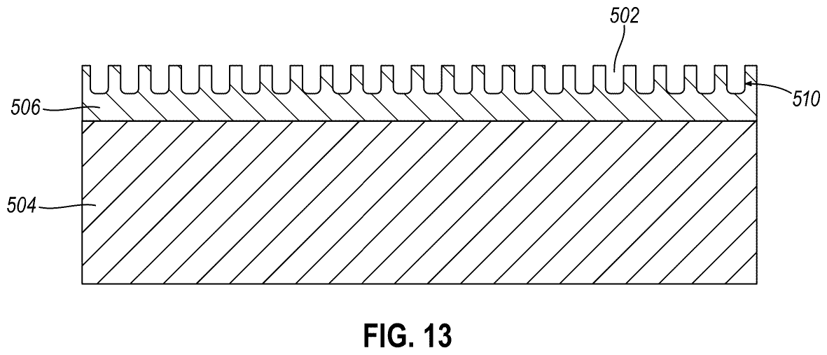

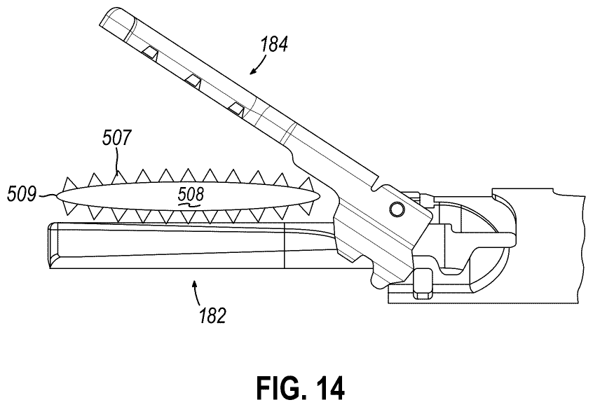

As will be described in greater detail below with reference to FIGS. 13 and 14, tissue contacting surfaces may include one or more removed portions (502) of any applied coating layer (506) to reduce sticking or otherwise promote tissue release while also improving the burst pressure of tissue samples prepared by tissue contacting surface. While removed portions (502) are described with reference to being applied to electrode surfaces of electrodes (194, 196), removed portions (502) can also be formed on an ultrasonic blade or another suitable surface that received a coating layer. As previously described, electrodes (194, 196) may be configured to cooperate to apply bipolar RF energy to tissue.

It is envisioned that removed portions (502) may be applied to select portions of the tissue contacting surfaces. Alternatively, removed portions (502) may be applied to the entire tissue contacting surface. In some versions, removed portions (502) may be applied to the entire outer surface of electrodes (194, 196) or the entire outer surface of an ultrasonic blade. In other versions, removed portions (502) may be applied to only select outer surfaces or to select portions of select outer surfaces of electrodes (194, 196) or select outer surfaces of an ultrasonic blade that experience sticking or high-pressure during tissue clamping. Removed portions (502) may be disposed on a metallic surface of the tissue contacting surface.

### B. Coating Methods

In one or more versions, a coating deposition method, such as, but not limited to, any form of chemical vapor deposition and/or any form of physical vapor deposition, such as, but not limited to, plasma vapor deposition (PVD), plasma enhanced chemical vapor deposition (PECVD), and/or atomic layer deposition (ALD). PECVD is a conformal coating process performed in a vacuum chamber which can apply a coating to any exposed areas of a substrate to the plasma/vapor during deposition. Plasma is used to activate precursor gases, allowing for lower deposition temperatures and higher deposition rates compared to conventional chemical vapor deposition techniques.

In one or more versions, PECVD may be utilized to apply a polymeric coating. In one or more versions, polymeric coatings can include polydimethylsiloxane-like (PDMS-like) coatings, polytetrafluoroethylene-like (PTFE-like) coatings, fluoropolymer coatings, silicone dip or spray coatings, and/or parylene coatings as coating layer (506). In one version, the coating is PDMS. PECVD shows very promising tissue anti-sticking results, however, the burst performance of thicker PDMS-like coatings (i.e., wherein the thickness is greater than 350 nm) at initial burst cycles can be low. In one or more versions, burst performance is determined by sealing and cutting tissue with instrument (100) discussed above, and then testing the performance of that seal versus varying amount of pressure. The greater burst performance equates to a better seal.

### C. Formation of Removed Portions

To address the low burst performance, portions of coating (506) applied during PECVD can be selectively removed as shown in FIG. 13. In one or more versions, the selective removal of portions of coating (506) can allow for the RF energy to pass through or be less impacted by coating (506) and as a result, the burst performance of a seal formed by instrument (100) containing coating (506) with removed portions (502) is improved as compared to the burst performance of a seal formed by instrument (100) containing coating (506) without removed portions (502). However, there is fine balance between improving the burst performance while maintaining the tissue anti-sticking performance of applied coating (506).

In one or more versions, portions of coating (506) applied during PECVD can be selectively removed by mechanically applying abrasion in a patterned or unpatterned formation to simulate the effects of coating (506) “break-in”, creating removed portions (502) in the form of microchannels as shown in FIG. 14. In one version, abrasion is applied by leveraging a medium (508) that the jaws (182, 184) of end effector (180) can be clamped on post application of coating (506). In practice, medium (508) contains abrasive elements (507) embedded on a surface (509) in a targeted pattern. In one version, abrasive elements (507) embedded on surface (509) of medium (508) can be diamonds.

In one or more versions, the act of clamping device (100) onto medium (508) creates penetration into coating (506) which creates removed portions (502) in the pattern (or unpatterned) form base on the way in which abrasive elements (507) are positioned in and shaped onto surface (509). The ability to position abrasive elements (507) into surface (509) allows for feature size and pattern tailoring of removed portions (502).

In one or more versions, the process of selectively removing coating (506) takes place within a different vacuum chamber than the PECVD takes place. In one or more versions, PECVD can be applied in a batch process wherein multiple components are coated simultaneously and in one or more versions, the process of selectively removing coating (506) can occur in a batch process wherein multiple components have coating (506) removed simultaneously.

### D. Characteristics of Removed Portions

By controlling the size and depth of each removed portion (502) relative to base substrate (504), removed portions (502) may decrease the amount of tissue sticking compared to base substrates (504) having generally smooth surfaces. For example, removed portions (502) may reduce tissue sticking compared to base substrates (504), which may reduce the number of protein bonding sites.

In one or more versions, each removed portion (502) has a removal depth of less than an entirety of a coating application thickness of applied coating (506) such that each removed portion (502) does not reach down to substrate (504). In yet other versions, each removed portion (502) has a removal depth equal to the coating application thickness of applied coating (506), such that each removed portion (502) reaches down to substrate (504). In one or more versions, the removal depth of each removed portion (502) can be from about 50% to about 100% of the coating application thickness of coating (506). In yet other versions, the removal depth of each removed portion (502) may be from about 50% to about 90% of the coating application thickness of coating (506), from about 50% to about 80%, from about 50% to about 70%, and from about 50% to about 70%. In one or more versions, the removal depth of each removed portion (502) can have any value between any of the foregoing ranges.

In one or more versions, from about 10% to about 50% of applied coating (506) can be removed. In yet other versions, from about 10% to about 40% of applied coating (506) can be removed, from about 10% to about 30%, and from about 10% to about 20%. In one or more versions, the percentage of applied coating (506) removed can have any value between any of the foregoing ranges. In one or more versions, the percentage of applied coating (506) removed can be adjusted by increasing the number of removed portions (502), by increasing removal depth of each removed portion (502), by increasing the diameter of each removed portion (502), or combinations thereof.

Although removed portions (502) as shown in FIG. 13 have been removed in patterned formation (510), yet other versions removed portions (502) can be removed in an un-patterned formation. Although each removed portion (502) as shown in FIG. 13 is shown as a circular dot pattern (510), in yet other versions each removed portion (502) can take the form of other shapes such as, but not limited to circles, squares, rectangles, diamonds, or stars.

In one or more versions, coating layer (506) can have a coating application thickness (CT) of between about 200 nm and about 300 nm, between about 220 nm and about 280 nm, or between about 240 nm and about 260 nm. In one version, coating layer (506) may have a coating application thickness (CT) greater than 300 nm. In one or more versions, coating layer (506) can have a coating application thickness between any of the foregoing ranges.

In one or more versions, the diameter of each removed portion (502) can be from about 0.001 inches to about 0.010 inches, from 0.002 inches to about 0.008 inches, or from about 0.004 inches to about 0.006 inches. In one or more versions, the diameter of each removed portion (502) can have any value between any of the foregoing ranges. In one version, the diameter of each removed portion (502) is about 0.005 inches.

### E. Characteristics of Abrasion Medium

In one or more versions, medium (508) may be selected from material such as steel, foam, plastic, sandpaper, or combinations thereof. In one version, medium (508) is steel to maximize force. In yet another version, medium (508) is foam to limit force application of abrasive elements (507). In one or more versions, the hardness of medium (508) is from 1 MOH to 9 MOH. In one or more versions, abrasive elements (507) may be selected from material such as diamonds, emery, garnet, silica, alumina silicate, lime, chalk, silicon carbide, aluminum oxide, ceramic, alumina, and alumina-zirconia.

In one or more versions, medium (508) may be secondarily activated via vibration to enhance penetration of abrasive elements (507) and to control the load applied during clamping to achieve the desired effect of dimpling or complete break-in.

### V. Coating Removal via Rolling of Abrasive Material

### A. Overview

Instruments, such as instrument (100) may generate heat as end effectors, such as end effector (180), to seal and/or cut tissue. Tissue contacting surfaces of the instruments may tend to stick to the treated tissue. The tissue contacting surfaces are intended to include at least one of an ultrasonic blade, electrodes (194, 196), or another suitable tissue contacting surface The tissue contacting surfaces include a base surface that is configured to contact the tissue. For example, base surfaces may include, for example, an outer surface of an ultrasonic blade or an electrode surface of electrodes (194, 196). Tissue sticking may cause reduced surgical efficiency.

The issue of tissue sticking is typically overcome by the addition of a coating layer to the tissue contacting surfaces of an instrument. However, the addition of a coating layer to tissue contacting surfaces may lead to tissue cut by the instrument to have low burst pressure. To improve the burst pressure while maintaining tissue anti-sticking performance, the present disclosure removes portions of the applied coating.

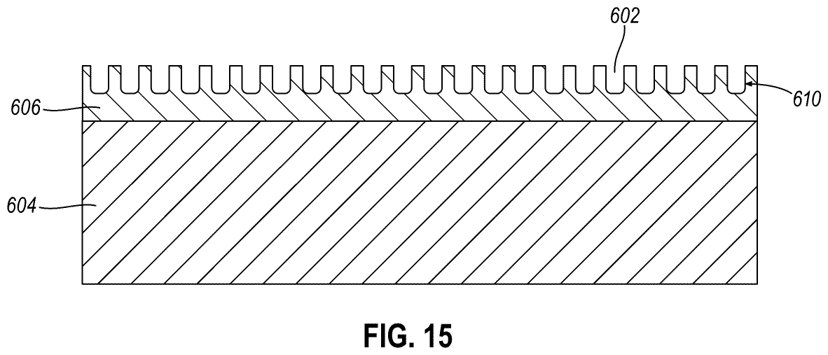

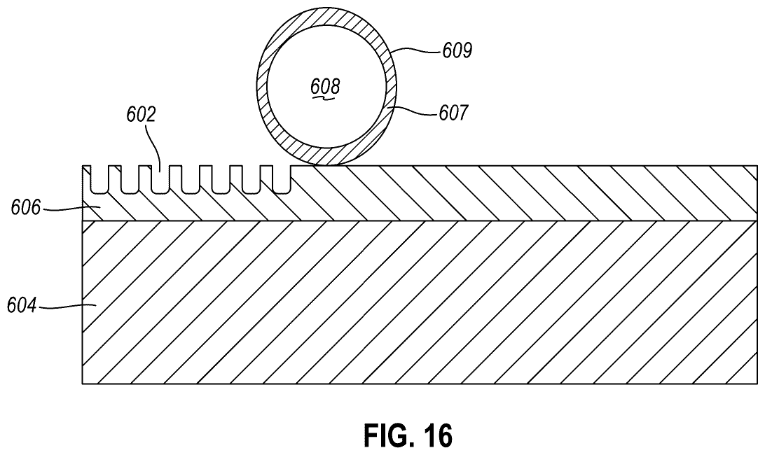

As will be described in greater detail below with reference to FIGS. 15 and 16, tissue contacting surfaces may include one or more removed portions (602) of any applied coating layer (606) to reduce sticking or otherwise promote tissue release while also improving the burst pressure of tissue samples prepared by tissue contacting surfaces. While removed portions (602) are described with reference to being applied to electrode surfaces of electrodes (194, 196), removed portions (602) can also be formed on an ultrasonic blade or another suitable surface that received a coating layer. As previously described, electrodes (194, 196) may be configured to cooperate to apply bipolar RF energy to tissue.

It is envisioned that removed portions (602) may be applied to select portions of the tissue contacting surfaces. Alternatively, removed portions (602) may be applied to the entire tissue contacting surface. In some versions, removed portions (602) may be applied to the entire outer surface of electrodes (194, 196) or the entire outer surface of an ultrasonic blade. In other versions, removed portions (602) may be applied to only select outer surfaces or to select portions of select outer surfaces of electrodes (194, 196) or select outer surfaces of an ultrasonic blade that experience sticking or high-pressure during tissue clamping. Removed portions (602) may be disposed on a metallic surface of the tissue contacting surface.

### B. Coating Methods

In one or more versions, a coating deposition method, such as, but not limited to, any form of chemical vapor deposition and/or any form of physical vapor deposition, such as, but not limited to, plasma vapor deposition (PVD), plasma enhanced chemical vapor deposition (PECVD), and/or atomic layer deposition (ALD). PECVD is a conformal coating process performed in a vacuum chamber which can apply a coating to any exposed areas of a substrate to the plasma/vapor during deposition. Plasma is used to activate precursor gases, allowing for lower deposition temperatures and higher deposition rates compared to conventional chemical vapor deposition techniques.

In one or more versions, PECVD may be utilized to apply a polymeric coating. In one or more versions, polymeric coatings can include polydimethylsiloxane-like (PDMS-like) coatings, polytetrafluoroethylene-like (PTFE-like) coatings, fluoropolymer coatings, silicone dip or spray coatings, and/or parylene coatings as coating layer (606). In one version, the coating is PDMS. PECVD shows very promising tissue anti-sticking results, however, the burst performance of thicker PDMS-like coatings (i.e., wherein the thickness is greater than 350 nm) at initial burst cycles can be low. In one or more versions, burst performance is determined by sealing and cutting tissue with instrument (100) discussed above, and then testing the performance of that seal versus varying amount of pressure. The greater burst performance equates to a better seal.

### C. Formation of Removed Portions

To address the low burst performance, portions of coating (606) applied during PECVD can be selectively removed as shown in FIG. 15. In one or more versions, the selective removal of portions of coating (606) can allow for the RF energy to pass through or be less impacted by coating (606) and as a result, the burst performance of a seal formed by instrument (100) containing coating (606) with removed portions (602) is improved as compared to the burst performance of a seal formed by instrument (100) containing coating (606) without removed portions (602). However, there is fine balance between improving the burst performance while maintaining the tissue anti-sticking performance of applied coating (606).

In one or more versions, portions of coating (606) applied during PECVD can be selectively by rolling a medium (608) containing abrasive elements (607) over a surface to simulate the effects of coating (606) “break-in”, creating removed portions (602) as shown in FIG. 16. In one version, abrasion is applied by rolling a medium (608) over a jaw, such as jaws (182, 184) of end effector (180), after application of coating (606). In practice, medium (608) contains abrasive elements (607) embedded on a surface (609) in a targeted patterned or unpatterned formation. In one version, abrasive elements (607) embedded on surface (609) of medium (608) can be diamonds, silicon carbide, sharp stainless-steel particles, or combinations thereof.

In one or more versions, the act of rolling medium (608) on elements of device (100) creates penetration into coating (606) which creates removed portions (602) in the pattern (or unpatterned) form based on the way in which abrasive elements (607) are positioned in and shaped onto surface (609). The ability to position abrasive elements (607) into surface (609) allows for feature size and pattern tailoring of removed portions (602).

In one or more versions, the process of selectively removing coating (606) takes place within a different vacuum chamber than the PECVD takes place. In one or more versions, PECVD can be applied in a batch process wherein multiple components are coated simultaneously and in one or more versions, the process of selectively removing coating (606) can occur in a batch process wherein multiple components have coating (606) removed simultaneously.

### D. Characteristics of Removed Portions

By controlling the size and depth of each removed portion (602) relative to base substrate (604), removed portions (602) may decrease the amount of tissue sticking compared to base substrates (604) having generally smooth surfaces. For example, removed portions (602) may reduce tissue sticking compared to base substrates (604), which may reduce the number of protein bonding sites.

In one or more versions, each removed portion (602) has a removal depth of less than an entirety of a coating application thickness of applied coating (606) such that each removed portion (602) does not reach down to substrate (604). In yet other versions, each removed portion (602) has a removal depth equal to the coating application thickness of applied coating (606), such that each removed portion (602) reaches down to substrate (604). In one or more versions, the removal depth of each removed portion (602) can be from about 50% to about 100% of the coating application thickness of coating (606). In yet other versions, the removal depth of each removed portion (602) may be from about 50% to about 90% of the coating application thickness of coating (606), from about 50% to about 80%, from about 50% to about 70%, and from about 50% to about 70%. In one or more versions, the removal depth of each removed portion (602) can have any value between any of the foregoing ranges.

In one or more versions, from about 10% to about 50% of applied coating (606) can be removed. In yet other versions, from about 10% to about 40% of applied coating (606) can be removed, from about 10% to about 30%, and from about 10% to about 20%. In one or more versions, the percentage of applied coating (606) removed can have any value between any of the foregoing ranges. In one or more versions, the percentage of applied coating (606) removed can be adjusted by increasing the number of removed portions (602), by increasing removal depth of each removed portion (602), by increasing the diameter of each removed portion (602), or combinations thereof.

Although removed portions (602) as shown in FIG. 15 have been removed in patterned formation (610), yet other versions removed portions (602) can be removed in an un-patterned formation. Although each removed portion (602) as shown in FIG. 15 is shown as a circular dot pattern (610), in yet other versions each removed portion (602) can take the form of other shapes such as, but not limited to circles, squares, rectangles, diamonds, or stars.

In one or more versions, coating layer (606) can have a coating application thickness (CT) of between about 200 nm and about 300 nm, between about 220 nm and about 280 nm, or between about 240 nm and about 260 nm. In one version, coating layer (606) may have a coating application thickness (CT) of greater than 300 nm. In one or more versions, coating layer (606) can have a coating application thickness between any of the foregoing ranges.

In one or more versions, the diameter of each removed portion (602) can be from about 0.001 inches to about 0.010 inches, from 0.002 inches to about 0.008 inches, or from about 0.004 inches to about 0.006 inches. In one or more versions, the diameter of each removed portion (602) can have any value between any of the foregoing ranges. In one version, the diameter of each removed portion (602) is about 0.005 inches.

### E. Characteristics of Abrasion Medium

In one or more versions, medium (608) may be selected from material such as steel, foam, plastic, sandpaper, or combinations thereof. In one version, medium (608) is steel to maximize force. In yet another version, medium 6508) is foam to limit force application of abrasive elements (607). In one or more versions, the hardness of medium (608) is from 1 MOH to 9 MOH. In one or more versions, abrasive elements (607) may be selected from material such as diamonds, emery, garnet, silica, alumina silicate, lime, chalk, silicon carbide, aluminum oxide, ceramic, alumina, and alumina-zirconia.

In one or more versions, medium (608) may be secondarily activated via vibration to enhance penetration of abrasive elements (607) and to control the load applied during rolling to achieve the desired effect of dimpling or complete break-in.

### IV. Illustrative Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method of manufacturing a surgical instrument that includes a tissue contacting surface operable to apply ultrasonic energy or RF energy to tissue, the method comprising (a) applying a hydrophobic coating that includes silicone to a base surface of the tissue contacting surface to form an applied coating layer having a coating application thickness; and (b) removing portions of the applied coating layer with a resiliently biased mechanical coating removal tool to form a plurality of removed portions of coating, wherein a removal depth of each removed portion of the plurality of removed portions can each be from 50% to 100% of the coating application thickness such that from 10% to 50% of the applied coating layer is removed from the base surface.

### Example 2

The method of Example 1, wherein the act of applying utilizes plasma enhanced chemical vapor deposition (PECVD) to form the applied coating layer.

### Example 3

The method of Example 1 or 2, wherein the hydrophobic coating is a polydimethylsiloxane-like (PDMS-like) coating, a polytetrafluoroethylene-like (PTFE-like) coating, a fluoropolymer coating, a silicone dip or spray coating, and/or a parylene coating.

### Example 4

The method of one or more of Example 1 through 3, wherein the resiliently biased mechanical coating removal tool is a drag bit.

### Example 5

The method of one or more of Example 1 through 4, wherein the plurality of removed portions are selectively removed to form a pattern of removed portions.

### Example 6

The method of Example 5, wherein the plurality of removed portions form a pattern of lines, and wherein each line of the pattern of lines has a diameter of from about 0.001 inches to about 0.010 inches.

### Example 7

The method of one or more of Example 1 through 6, wherein the removal depth of each removed portion of the plurality of removed portions is less than the coating application thickness of the applied coating layer.

### Example 8

The method of one or more of Example 1 through 6, wherein the removal depth of each removed portion of the plurality of removed portions is equal to the coating application thickness of the applied coating layer.

### Example 9

The method of one or more of Example 1 through 8, further comprising: (a) loading the tissue contacting surface into a vacuum chamber; (b) decreasing a pressure of the vacuum chamber; and (c) plasma treating the base surface after decreasing the pressure of the vacuum chamber to clean and activate the tissue contacting surface.

### Example 10

The method of Example 9, wherein the act of plasma treating is performed prior to the act of applying the hydrophobic coating that includes the silicone and uses at least one of oxygen or argon.

### Example 11

A method of manufacturing a surgical instrument that includes a tissue contacting surface operable to apply ultrasonic energy or RF energy to tissue, the method comprising: (a) loading the tissue contacting surface into a vacuum chamber; (b) decreasing a pressure of the vacuum chamber; (c) plasma treating at least one surface of the tissue contacting surface after decreasing the pressure of the vacuum chamber to clean and activate the tissue contacting surface; (d) applying a hydrophobic coating that includes silicone to the at least one surface of the tissue contacting surface to form an applied coating layer having a coating application thickness; and (e) removing portions of the applied coating layer with a resiliently biased mechanical coating removal tool to form a plurality of removed portions of coating, wherein a removal depth of each removed portion of the plurality of removed portions can each be from 50% to 100% of the coating application thickness such that from 10% to 50% of the applied coating layer is removed from the base surface.

### Example 12

The method of Example 11, wherein the act of applying utilizes plasma enhanced chemical vapor deposition (PECVD) to form the applied coating layer.

### Example 13

The method of Example 11 or 12, wherein the hydrophobic coating is a polydimethylsiloxane-like (PDMS-like) coating, a polytetrafluoroethylene-like (PTFE-like) coating, a fluoropolymer coating, a silicone dip or spray coating, and/or a parylene coating.

### Example 14

The method of one or more of Examples 11 through 13, wherein the resiliently biased mechanical coating removal tool is a drag bit.

### Example 15

The method of one or more of Examples 11 through 14, wherein the removal depth of each removed portion of the plurality of removed portions is less than the coating application thickness of the applied coating layer.

### Example 16

The method of one or more of Examples 11 through 14, wherein the removal depth of each removed portion of the plurality of removed portions is equal to the coating application thickness of the applied coating layer.

### Example 17

A surgical instrument comprising: (a) a shaft assembly; and (b) an end effector extending distally from the shaft assembly, wherein the end effector includes a tissue contacting surface configured to apply energy to treat tissue, wherein the tissue contacting surface includes at least one of an ultrasonic blade or an electrode, the tissue contacting surface comprising: (i) a base surface configured to contact the tissue, (ii) a hydrophobic coating layer that includes silicone on the base surface having a coating application thickness, and (iii) a plurality of removed portions of the hydrophobic coating layer removed by a resiliently biased mechanical coating removal tool, wherein a removal depth of each removed portion of the plurality of removed portions can each be from 50% to 100% of the coating application thickness such that from 10% to 50% of the applied coating layer is removed from the base surface.

### Example 18

The surgical instrument of Example 17, wherein the removal depth of each removed portion of the plurality of removed portions is less than the coating application thickness of the hydrophobic coating layer.

### Example 19

The surgical instrument of Example 17, wherein the removal depth of each removed portion of the plurality of removed portions is equal to the coating application thickness of the hydrophobic coating layer.

### Example 20

The surgical instrument of one or more of Examples 16 through 19, wherein the hydrophobic coating layer is a polydimethylsiloxane-like (PDMS-like) coating layer, a polytetrafluoroethylene-like (PTFE-like) coating layer, a fluoropolymer coating layer, a silicone dip or spray coating layer, and/or a parylene coating layer.

### V. Miscellaneous

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those of ordinary skill in the art.

While the examples herein are described mainly in the context of electrosurgical instruments, it should be understood that various teachings herein may be readily applied to a variety of other types of devices. By way of example only, the various teachings herein may be readily applied to other types of electrosurgical instruments, tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those of ordinary skill in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCITM system by Intuitive Surgical, Inc., of Sunnyvale, California. Similarly, those of ordinary skill in the art will recognize that various teachings herein may be readily combined with various teachings of U.S. Pat. No. 6,783,524, entitled “Robotic Surgical Tool with Ultrasound Cauterizing and Cutting Instrument,” published Aug. 31, 2004, the disclosure of which is incorporated by reference herein, in its entirety.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. A method of manufacturing a surgical instrument that includes a tissue contacting surface operable to apply radio frequency (RF) energy to tissue, the method comprising:
(a) applying a hydrophobic coating that includes silicone to a base surface of the tissue contacting surface to form an applied coating layer having a coating application thickness; and
(b) removing portions of the applied coating layer with a resiliently biased mechanical coating removal tool to form a plurality of removed portions of coating,
wherein a removal depth of each removed portion of the plurality of removed portions can each be from 50% to 100% of the coating application thickness such that from 10% to 50% of the applied coating layer is removed from the base surface.

2. The method of claim 1, wherein the act of applying utilizes plasma enhanced chemical vapor deposition (PECVD) to form the applied coating layer.

3. The method of claim 1, wherein the hydrophobic coating is a polydimethylsiloxane-like (PDMS-like) coating, a polytetrafluoroethylene-like (PTFE-like) coating, a fluoropolymer coating, a silicone dip or spray coating, and/or a parylene coating.

4. The method of claim 1, wherein the resiliently biased mechanical coating removal tool is a drag bit.

5. The method of claim 1, wherein the plurality of removed portions are selectively removed to form a pattern of removed portions.

6. The method of claim 5, wherein the plurality of removed portions form a pattern of lines, and wherein each line of the pattern of lines has a diameter of from about 0.001 inches to about 0.010 inches.

7. The method of claim 1, wherein the removal depth of each removed portion of the plurality of removed portions is less than the coating application thickness of the applied coating layer.

8. The method of claim 1, wherein the removal depth of each removed portion of the plurality of removed portions is equal to the coating application thickness of the applied coating layer.

9. The method of claim 1, further comprising:
(a) loading the tissue contacting surface into a vacuum chamber;
(b) decreasing a pressure of the vacuum chamber; and
(c) plasma treating the base surface after decreasing the pressure of the vacuum chamber to clean and activate the tissue contacting surface.

10. The method of claim 9, wherein the act of plasma treating is performed prior to the act of applying the hydrophobic coating and uses at least one of oxygen or argon.

11. A method of manufacturing a surgical instrument that includes a tissue contacting surface operable to apply ultrasonic energy or RF energy to tissue, the method comprising:
(a) loading the tissue contacting surface into a vacuum chamber;
(b) decreasing a pressure of the vacuum chamber;
(c) plasma treating at least one surface of the tissue contacting surface after decreasing the pressure of the vacuum chamber to clean and activate the tissue contacting surface;
(d) applying a hydrophobic coating that includes silicone to the at least one surface of the tissue contacting surface to form an applied coating layer having a coating application thickness; and
(e) removing portions of the applied coating layer with a resiliently biased mechanical coating removal tool to form a plurality of removed portions of coating, wherein a removal depth of each removed portion of the plurality of removed portions can each be from 50% to 100% of the coating application thickness such that from 10% to 50% of the applied coating layer is removed from the base surface.

12. The method of claim 11, wherein the act of applying utilizes plasma enhanced chemical vapor deposition (PECVD) to form the applied coating layer.

13. The method of claim 11, wherein the hydrophobic coating is a polydimethylsiloxane-like (PDMS-like) coating, a polytetrafluoroethylene-like (PTFE-like) coating, a fluoropolymer coating, a silicone dip or spray coating, and/or a parylene coating.

14. The method of claim 11, wherein the resiliently biased mechanical coating removal tool is a drag bit.

15. The method of claim 11, wherein the removal depth of each removed portion of the plurality of removed portions is less than the coating application thickness of the applied coating layer.

16. The method of claim 11, wherein the removal depth of each removed portion of the plurality of removed portions is equal to the coating application thickness of the applied coating layer.

17. A surgical instrument comprising:
(a) a shaft assembly; and
(b) an end effector extending distally from the shaft assembly, wherein the end effector includes a tissue contacting surface configured to apply energy to treat tissue, wherein the tissue contacting surface includes at least one of an ultrasonic blade or an electrode, the tissue contacting surface comprising:
(i) a base surface configured to contact the tissue,
(ii) a hydrophobic coating layer that includes silicone on the base surface having a coating application thickness, and
(iii) a plurality of removed portions of the hydrophobic coating layer removed by a resiliently biased mechanical coating removal tool,
wherein a removal depth of each removed portion of the plurality of removed portions can each be from 50% to 100% of the coating application thickness such that from 10% to 50% of the applied coating layer is removed from the base surface.

18. The surgical instrument of claim 17, wherein the removal depth of each removed portion of the plurality of removed portions is less than the coating application thickness of the hydrophobic coating layer.

19. The surgical instrument of claim 17, wherein the removal depth of each removed portion of the plurality of removed portions is equal to the coating application thickness of the hydrophobic coating layer.

20. The surgical instrument of claim 19, wherein the hydrophobic coating layer is a polydimethylsiloxane-like (PDMS-like) coating layer, a polytetrafluoroethylene-like (PTFE-like) coating layer, a fluoropolymer coating layer, a silicone dip or spray coating layer, and/or a parylene coating layer.
